(19) United States
(12) Patent Application Publication
(10) Pub. No.: US 2005/0049224 A1
(43) Pub. Date: Mar. 3, 2005

(54) Antimicrobial mixtures comprising iodopropynyl compounds and dimethyldithiocarbamate derivatives

(76) Inventors: Kamlesh D. Gaglani, Belle Mead, NJ (US); Roger Errol Smith, Neshanic Station, NJ (US); Gordhandas G. Thakkar, Edison, NJ (US)

Correspondence Address: Robert F. Tavares;c/o Troy Corporation, 8 Vreeland Road, Florham Park, NJ, 07932-0955, US

(21) Appl. No.: 10/652,524

(22) Filed: Aug. 29, 2003

(51) Int. Cl. A61K 31/724; A61K 31/325

(52) U.S. Cl. 514/58; 514/479

(57) ABSTRACT

This invention is directed to an synergistic antimicrobial composition which comprises a mixture of an iodopropynyl compound and a dimethyldithiocarbamate compound, said mixture provided in an amount sufficient to protect a substrate from attack by one or more microbial organisms. The composition can be used broadly in industrial systems and more particularly with substrates such as paints, coatings, plastics, wood and wood composites.

### BACKGROUND OF THE INVENTION

1. Field of the Invention

The present invention pertains to novel antimicrobial compositions suitable for use in the protection of substances that are subject to attack, infestation and spoilage resulting from the growth of microorganisms such as fungi, bacteria, algae etc. This invention is also directed to the protection of substances that require high temperatures in their processing including, but not restricted to, wood composites and plastics such as polyvinyl chloride (PVC), low density polyethylene (LDPE), low density polyethylene foam, plastisols, and polyurethane.

2. Description of the Background

It is generally known that many materials, which come in contact with moisture, are prone to attack, defacement, spoilage, infestation and various kinds of destruction by a variety of species of microorganisms including fungi, yeast, algae and bacteria. As a result, there has always been a great need for effective and economical means to protect, for extended periods of time, commercial substances, articles of commerce and formulations from such deterioration and destruction.

Materials which may need such protection include, for example, plastics, wood, wood products, wood composites, plastic-wood composites, molded plastics, building materials, paper, toys, roof coatings, paints, other coating formulations, surfactants, proteins, starch-based compositions, inks, emulsions, resins, stucco, concrete, stone, wood adhesives, caulking, sealants, leather, leather finishes, soap wrappers, packaging materials, spin finishes, fabrics, cordage, carpet backings, electrical insulation, and medical devices. Additional materials that are prone to degradation by microorganisms include polymeric films and articles, polymer dispersions or aqueous latex paints containing polyvinyl alcohol, polyacrylates or vinylpolymers, thickener solutions containing cellulose derivatives, clay and mineral slurries, metal working fluids and optical brighteners.

Both wet state compositions and dry films can be attacked by objectionable microorganisms that can spoil and significantly impair their usefulness. Such degradation may produce, inter alia, changes in pH values, gas formation, defacement, discoloration, the formation of objectionable odors, and/or changes in rheological properties.

A great deal of effort has gone into developing a wide variety of materials which, to various degrees, are effective in retarding or preventing the growth of such microorganisms in a variety of circumstances. Such antimicrobial compounds have included halogenated compounds, organometallic compounds, quaternary ammonium compounds, phenolics, metallic salts, heterocyclic amines, formaldehyde donors, organosulfur compounds and the like.

No single organic antimicrobial compound has been able to provide protection against all microorganisms or has been suitable for all applications. In addition to limitations concerning efficacy, other limitations may restrict the usefulness of certain antimicrobials for certain applications. Considerations such as stability, physical properties, toxicological profile, regulatory considerations, economic considerations or environmental concerns may render a particular ingredient unsuitable for a particular use. Antimicrobials which are suitable in many applications may not, for example, be able to survive the harsh processing conditions that may be required in the formation of plastic materials where the processing temperatures can reach 300° F. to 400° F. There is, therefore, a need to constantly develop new combinations that will provide adequate protection in a variety of applications and under a variety of conditions.

A judicious choice of combinations may provide a way to maximize benefits and minimize problems. Ideally, a combination wherein the antimicrobial activity is enhanced while the less desirable properties are suppressed can provide an unexpectedly superior product. The task is to find combinations that will provide protection against a wide variety of problem microorganisms, will not adversely affect the product to be protected, will maintain its integrity for an extended period of time, will not have any adverse effect on health or the environment and will meet the economic restraints that such an application might impose.

A number of iodopropynyl compounds are known to have antimicrobial activity. The most common and most commercially successful of these is the antimicrobial 3-iodo-2-propynyl butyl carbamate (IPBC) which has outstanding fungicidal properties and has found wide use in a number of applications as an effective antimicrobial preservative, both for wet and dry films. More recently, it has been found that inclusion complexes can be formed between IPBC with α-cyclodextrins. U.S. Pat. No. 5,906,981, which is directed to such complexes, also teaches that such complexes can be made from modified α-cyclodextrins. Thus by modifying the α-cyclodextrins, a number of IPBC based antimicrobial complexes may be provided which, due to the modifications made on the α-cyclodextrins, have allowed IPBC to be used under conditions and for applications that would not have been considered suitable for uncomplexed IPBC.

It is also known that dimethyldithiocarbamoyl derivatives and the salts thereof have useful antimicrobial properties. Two of the most commonly used are zinc bis(dimethyldithiocarbamate) known as Ziram and bis(dimethylthiocarbamoyl) disulfide known as Thiram. Both find extensive use as fungicides in agriculture. In addition, sodium N-dimethyldithiocarbamate is a known antimicrobial used as a slimicide and for formulations used in the paper making industry.

Combinations comprising 3-iodo-2-propynyl butyl carbamate and dimethyldithiocarbamoyl compounds for the control of microorganisms, have not been reported.

### SUMMARY OF THE INVENTION

The present invention is directed to certain antimicrobial mixtures comprising an iodopropynyl compound and a dimethyldithiocarbamoyl compound. For some applications, the iodopropynyl compound is in the form of an inclusion complex.

The present invention is also directed to methods for inhibiting microbial growth which comprises using said mixtures

### DETAILED DESCRIPTION OF THE INVENTION

One component of this invention is an iodopropynyl compound, preferably the commercially available 3-iodo-2-propynyl butyl carbamate (IPBC). A second component of the antimicrobial combination of this invention is a dimethyldithiocarbamate derivative, preferably the commercially available zinc bis(dimethyldithiocarbamate) (Ziram) or bis(dimethylthiocarbamoyl) disulfide (Thiram). In this specification, the term IPBC will be used for 3-iodo-2-propynyl butyl carbamate, the term Ziram will be used for zinc dimethyldithiocarbamate and the term Thiram will be used for bis-(dimethylthiocarbamoyl) disulfide.

In the preferred embodiments of this invention it has been found that synergistic results appear across all blends. The practical range of IPBC to dimethydithiocarbamate derivative runs from about 1:25 to 25:1, preferably about 1:9 to 9:1 with 3:7 to 7:3 being especially preferred.

The methods by which the formulations of this invention are used depend to some degree on the application involved. A person having ordinary skill in the art with the intended application will be able to incorporate said compositions into the materials to be protected. In most instances the compositions of this invention may be simply added by mixing or dispersing the active ingredients in a selected proportion with the other ingredients used to prepare the product to be protected. Depending on the intended use, said ingredients may include a diluent, emulsifiers, wetting agents, stabilizers and/or other adjuvants that are necessary or desirable in preparing the final formulation. Such adjuvants may include organic binding agents, additional antimicrobials, fillers such as CaCO₃, talc, TiO₂, pigments, auxiliary solvents, processing additives, fixatives, plasticizers, UV-stabilizers, stability enhancers, water soluble or water insoluble dyes, color pigments, siccatives, corrosion inhibitors, antisettlement agents, anti-skinning agents and the like.

Depending on the final product to be protected, the levels of the antimicrobial compositions of this invention in that final product can vary over a broad range of concentration ranging from 0.004% to 2.0%. The preferred useful range is between about 0.1 to 1.0% in said final formulations.

In applications wherein the α-cyclodextrin inclusion complex of IPBC is to be used, similar methods can be used for adding said antimicrobials to the substrate to be protected as were used for adding the pure IPBC except that the amounts of complex added will have to be adjusted to insure that the proper level of IPBC, per se, will be incorporated into the final formulation. Again, it is well within the skill of the ordinary practitioner familiar with the formulation of biocides in the materials to be protected to incorporate said biocides in them.

The use of IPBC in the form of the α-cyclodextrin inclusion complex will be favored in those applications where the IPBC is to be incorporated into a material via a process in which high temperatures will be used during processing such as in the case of a plastic article or plastic film. Any α-cyclodextrin inclusion complex should be suitable in such instances, but it may be preferable to use a modified α-cyclodextrin inclusion complex of IPBC when it is desirable to modify the properties of the inclusion complex. Methods for preparing α-cyclodextrin inclusion complexes and modified α-cyclodextrin inclusion complexes of IPBC are described in U.S. Pat. No. 5,906,981.

Using the IPBC in the form of an α-cyclodextrin complex may also be preferred in other, non-thermal applications to take advantage of a particular inclusion complex. For example, it may be preferable to use IPBC in the form of an α-cyclodextrin inclusion complex to add additional stability to the IPBC to retard the potential for unwanted decomposition and/or yellowing or it may be desirable to use an inclusion complex of a modified α-cyclodextrin wherein the cyclodextrin has been modified to increase water solubility.

The combinations of this invention may be provided as solid mixtures in powder form, as concentrates in extruded LDPE, as wettable powders, dispersions, or in any other suitable product type which is desirable. In this regard, the composition of the present invention can be provided as a ready-for-use product in the form of powder blends, aqueous dispersion, oil dispersions, emulsions or as a concentrate, especially in a polymer matrix.

As mentioned earlier, the combinations of this invention display synergistic effects. A synergistic effect is generally regarded as the response of a mixture of two or more components that is greater than the sum of the response of the individual components. A mathematical approach for assessing synergy, as reported by F. C. Kull, P. C. Elisman, H. D. Sylwestrowicz and P. K. Mayer, in Applied Microbiology, 9.538 (1961).

The following examples are presented to illustrate and explain the invention. Unless otherwise indicated, all references to parts and percentages are based on weight.

### EXAMPLES

### Example 1

Activity Against Aspergillus Niger

Minimum Inhibitory Concentrations (MIC) of the biocides Thiram [bis(dimethylthiocarbamoyl) disulfide, CAS # 137-26-8], Ziram [zinc bis(dimethyldithiocarbamate), CAS # 137-30-4] and IPBC [3-iodo-2-propynyl butyl carbamate, CAS # 55406-53-6] and various mixtures thereof were determined against the fungus Aspergillus niger, (ATTC 6275) with the aid of an Autoplate 4000 spiral plater and spiral gradient endpoint software (SGE). The spiral plater and SGE software are available from Spiral Biotech, Inc., presently of Norwood Mass.

Spore suspensions were prepared by growing Aspergillus niger (ATCC 6275) on a malt agar slant (Difco malt agar) in an incubator for 1 week at the temperature of 28° C. Spores were removed from the Aspergillus niger slant by adding a small amount of phosphate buffer pH 7.0 (Thomas Scientific phosphate buffer & magnesium chloride, Lot # 023-0703) and scraping with a sterile nichrome wire loop. This process was repeated twice. Loosened spores were removed from the slant by aseptically pouring them into a sterile bottle containing 30 ml. of phosphate buffer and a volume of roughly 40 ml. of 6-mm, borosilicate glass beads. The bead bottle was shaken to disperse the spores and additional buffer was added to a final volume of 50 ml. Spore density was adjusted in distilled water blanks to that of a 0.5. McFarland nephelometer standard.

Combinations of Ziram, Thiram and IPBC were prepared by mixing the fungicides in various ratios. In this example and in the following examples, the source of Ziram used was Mergal® W 600 (98% technical grade marketed by Troy GmbH), the source of Thiram used was Mergal® W-800 (98% technical grade marketed by Troy GmbH), the source of uncomplexed IPBC was a 40% dispersion of IPBC (Polyphase® P-100) marketed by Troy Corporation as EX 634-2 in Europe (hereinafter also referred to as the IPBC dispersion) and the source of the complexed IPBC was Polyphase® 604 (marketed by Troy Corporation) which is an inclusion complex of IPBC and a hydroxypropyl α-cyclodextrin. [The hydroxypropyl α-cyclodextrin is available from American Maize-Products Company as Cavitron HPACD].

The test materials were prepared for MIC determination including samples of the pure materials (Ziram, Thiram, uncomplexed IPBC, complexed IPBC) as well as various mixtures of IPBC (complexed and uncomplexed) with Ziram and Thiram wherein the ingredient ratios are 1:1, 2:1 and 4:1. With the aid of the SGE Software the proper aqueous dilutions of each biocide or test blend were prepared and adjusted to a final volume of 10 ml. Then, 250 micro-liters of each biocide mixture was processed by the spiral plater.

The Autoplate 4000 mechanically applied mixtures of test fungicides to 150 MM malt agar plates using an exponential application gradient. Gradients of applied fungicides were allowed to completely diffuse into the surface of the agar (1 to 4 hours at room temperature 23° C.) before inoculating the plates with fungi.

Spiral gradient plates were inoculated with the test microorganisms in a radial pattern using a paper template, generated by the SGE software, as a guide.

Inoculated spiral gradient plates were incubated for 48 hours in a 28° C. incubator.

Following incubation, visible growth of the test organism was observed on a portion of the radial streak and ended where the concentration of biocide was high enough to prevent growth. This growth endpoint was used by the SGE software to compute the exact MIC expressed as parts per million (PPM) of active fungicide. Each MIC result was reported as the average of at least three replicates.

Data were analyzed by the procedure reported by F. C. Kull, P. C. Elisman, H. D. Sylwestrowicz and P. K. Mayer, in Applied Microbiology, 9:538 (1961) where:
Qₐ=the quantity of A used in a binary mixture that gives the desired effect.
Q_{A}=the quantity of A which when used alone gives the desired effect.
Q_{b}=the quantity of B used in a binary mixture that gives the desired effect.
Q_{B}=the quantity of B which when used alone gives the desired effect.

The synergistic index (SI) for each mixture was determined by the equation SI=Qₐ/Q_{A}+Q_{b}/Q_{B}. A synergistic Index of less than 1 indicated that the combination exhibited synergistic properties.

Results are provided in Tables 1a, 1b and 1c that show that the compositions of this invention exhibit synergistic properties. The results obtained for IPBC, Ziram mixtures are set forth in Table 1a. Similar results are shown in Table 1b for mixtures of Thiram and IPBC. Table 1c shows further that if the IPBC is used in the form of its α-cyclodextrin complex (Polyphase 604) that a synergistic result is also observed.

TABLE 1a
IPBC (A) and Ziram (B) Mixtures

| Ratio A:B | MIC A + B | Qₐ | Q_{A} | Q_{b} | Q_{B} | |
|---|---|---|---|---|---|---|
| W/W | ppm | ppm | ppm | ppm | Ppm | SI |
| 1:1 | 1.13 | 0.57 | 1.08 | 0.57 | 91.24 | 0.54 |
| 2:1 | 1.07 | 0.72 | 1.08 | 0.35 | 91.24 | 0.67 |
| 4:1 | 0.78 | 0.59 | 1.08 | 0.20 | 91.24 | 0.55 |
| 1:2 | 1.05 | 0.35 | 1.08 | 0.70 | 91.24 | 0.33 |
| 1:4 | 1.37 | 0.34 | 1.08 | 1.03 | 91.24 | 0.33 |

TABLE 1b
IPBC (A) and Thiram (B) Mixtures

| Ratio A:B | MIC A + B | Qₐ | Q_{A} | Q_{b} | Q_{B} | |
|---|---|---|---|---|---|---|
| W/W | (ppm) | ppm | ppm | (ppm) | (ppm) | SI |
| 1:1 | 0.87 | 0.52 | 1.08 | 0.52 | 26.73 | 0.50 |
| 2:1 | 1.05 | 0.70 | 1.08 | 0.35 | 26.73 | 0.66 |
| 4:1 | 0.77 | 0.58 | 1.08 | 0.19 | 26.73 | 0.55 |
| 1:2 | 0.98 | 0.32 | 1.08 | 0.66 | 26.73 | 0.32 |
| 1:4 | 1.28 | 0.32 | 1.08 | 0.96 | 26.73 | 0.34 |

TABLE 1c
Complexed IPBC (A) and Ziram (B) Mixtures

| Ratio A:B | MIC A + B | Qₐ | Q_{A} | Q_{b} | Q_{B} | |
|---|---|---|---|---|---|---|
| W/W | (ppm) | (ppm) | (ppm) | (ppm) | (ppm) | SI |
| 1:1 | 0.23 | 0.12 | 0.18 | 0.12 | 91.24 | 0.67 |

### Example 2

Test Results in Paint

In this example an acrylic, vinyl acrylic white house paint was used as the test medium. The composition of the paint used in this example is shown in Table 2.

TABLE 2a
Formulation of Acrylic/Vinyl Acrylic White House Paint

| No | Ingredient | Supplier | % W/W |
|---|---|---|---|
| 1 | Natrosol 250 MHR 100% | Aqualon | 0.3 |
| 2 | Propylene glycol | | 1.7 |
| 3 | Tamol 850 (30%) | Rohm & Haas | 0.9 |
| 4 | KTPP | FMC | 0.12 |
| 5 | Nopco NXZ | Hüls | 0.1 |
| 6 | Triton CF-10 | Union Carbide | 0.21 |
| 7 | Water | | 13.44 |
| 8 | Titanium dioxide | Kerr McGee | 14.5 |
| 9 | Minex 4 | Uniman | 15.7 |
| 10 | Silica (Silver Bond B) | Uniman | 6.4 |
| 11 | Attagel | Engelhard | 0.85 |
| 12 | UCAR 379 | Union Carbide | 6.31 |
| 13 | Rhoplex AC-264 (60.5%) | Rohm & Haas | 25.2 |
| 14 | Nopco NXZ | Hüls | 0.17 |
| 15 | Propylene glycol | | 4.1 |
| 16 | Natrosol 250 MHR (2.5%) | | 10.0 |
| | TOTAL | | 100.00 |

Test formulations of the actives were prepared for each of the biocide blends to be tested (see tables 2b and 2c) by weighing the appropriate amounts of actives and adding them to the test paint so that in each test formulation the weight of the actives was 20% of the sample and 80% of the sample was paint. Each mixture was then stirred to achieve a homogeneous mixture. These test formulations were then incorporated into the test paint at the rate of 0.3% w/w, i.e. the final level of actives in the paint was 0.06%.

The test paints were then applied to a filter paper using a standard 3-mil drawdown bar to insure uniformity of the film. Samples were then dried at room temperature, and placed on agar contained in a Petri dish. The filter papers were then inoculated with a test inoculum of Aspergillus niger. Inoculated filter papers containing the test paints were placed in a constant temperature (28° C.) fungal incubator for four weeks. At the end of the incubation, fungal growth on each test paint was measured and recorded.

Results were recorded as fungus units determined by comparison to a standard chart with a range of 1 to 15 fungus units. A value of 15 indicates the painted square was totally covered with fungus. Values 5 through 15 showed gradations of growth from very little fungus growing on the surface to the total coverage of 15. A rating of 4 showed growth on the edges of the test square only, 3 showed growth up to the edges and 1 indicated no growth touching the test square.

The results are shown in Table 2b for IPBC+Ziram mixtures and in Table 2c for IPBC+Thiram mixtures. Each test blend was run at least 3 times and each result represents the average of the runs.

TABLE 2b

| TEST | IPBC % | Ziram % | Fungal Growth |
|---|---|---|---|
| 1 | 100 | 0 | 7.8 |
| 2 | 50 | 50 | 3.5 |
| 3 | 0 | 100 | 9.8 |
| 4 | 75 | 25 | 3.7 |

TABLE 2c

| TEST | IPBC % | Thiram % | Fungal Growth |
|---|---|---|---|
| 1 | 0 | 100 | 13.8 |
| 2 | 100 | 0 | 2.8 |
| 3 | 75 | 25 | 1.7 |
| 4 | 50 | 50 | 3.4 |
| 5 | 25 | 75 | 1.7 |

### Example 3

Test Results in PVC Film

In this example a PVC formulation for flexible vinyl film was used as the test medium. The composition of the flexible vinyl film is shown in Table 3a.

TABLE 3a
Formulation for Flexible Vinyl Film.

| Ingredient | Weight % |
|---|---|
| PVEC RESIN - OXY 230 | 65.7 |
| Plasticizer - DINP | 30.0 |
| Epoxidized Soya Bean Oil - ESO | 2.0 |
| Ba/Zn Stabilizer | 1.7 |
| Stearic Acid | 0.5 |
| Antimicrobial | 0.1 |

The antimicrobial blends were prepared by mixing the solid Ziram and a solid IPBC inclusion complex made in accordance with Example 1 of U.S. Pat. No. 5,906,981. Three antimicrobial blends were prepared wherein the ratios of Ziram to IPBC complex were 1:3, 1:1 and 3:1. Five blends were prepared by adding the appropriate amount of the biocide to separate vinyl film form formulations prepared according to Table 3a. The formulation was then thoroughly mixed and then processed using a two-roll mill at 370° F. to produce the plastic films. The plastic films containing the biocides that were produced were then cut into 1″×1″ squares which were used for antimicrobial testing in Examples 3A and 3B.

### Example 3A

Test Results Against Fungi

The method used for evaluating IPBC/Ziram blends for anti-fungal activity in plastic film was according to the methology described in ASTM G 21-90, “Standard Practice for Determining Resistance of Synthetic Polymeric Materials to Fungi.” The Fungi used in this method are a mixture of Aspergillus niger ATCC 9642, Penicillium pinophilum ATCC 11797, Chaetomium globosum ATCC 6205, Gliocaladium virens ATCC 9645, and Aureobasidium pullulans ATCC 9348. All cultures were maintained on Difco malt extract agar with the exception of Chaetomium, which was maintained on mineral salts agar with cellulose as carbon source and Penicillium pinophilum, which was maintained on Difco malt agar. Fungus cultures were maintained separately at 28° C. and sub cultured regularly.

Spores were removed from their growth slants by adding 10 ml of sterile pH 7.0 buffer (Thomas Scientific phosphate buffer & magnesium chloride, Lot #023-0703) containing 1 drop of Triton X 100 surfactant, and scraping with a sterile nichrome wire loop. This process was repeated twice for each culture. Loosened spores were aseptically decanted into a sterile container containing 45 ml of sterile phosphate buffer and a volume of 10 to 15 ml of 6-mm borosilicate glass beads. The container was shaken to break-up the fungus inoculum and release the spores from their mycelium. The fungus suspension was filtered through a thin layer of sterile glass wool to remove mycelial fragments. Equal volumes of spore suspensions from each fungus were blended together to make the test spore suspension. The test spore suspension was aseptically decanted into a sterilized hand sprayer for inoculation of samples.

Mineral salts agar was prepared as described in the ASTMG 21-90 procedure. The medium was modified to include 3% glucose as a carbon source to stimulate maximum growth of fungus during the test. The test medium was sterilized in an autoclave at 121° C. for 20 min. After cooling, the pH of the medium was adjusted between 6.0 to 6.5 using 0.01N NaOH solution.

Test plates were prepared by pouring 30 ml melted, sterile nutrient salts agar into sterile plastic Petri dishes.

After a brief (1 to 2 seconds) immersion in boiling water to kill bacterial contaminants, specimens were placed on the surface of the solidified agar near the center of the Petri plates. The entire surface of the Petri plate and sample was evenly inoculated by spraying-on test spore suspension. Inoculated plates were covered and incubated at 28° C. for three weeks. A scale of 0 to 4 was used to indicate the amount of growth covering the specimen where 0 indicated no growth, 1 indicated a trace of growth (<10%), 2 indicated light growth (30% to 60%) and 4 indicated heavy growth (>60%).

TABLE 3b

| Test Blend # | IPBC, ppm | Ziram, ppm | Fungal Growth |
|---|---|---|---|
| 1 | 0 | 1000 | 2 |
| 2 | 250 | 750 | 1 |
| 3 | 1000 | 0 | 0 |
| 4 | 0 | 1000 | 2 |
| 5 | 1000 | 0 | 0 |
| 6 | 500 | 500 | 0 |
| 7 | 500 | 500 | 0 |
| 8 | 750 | 250 | 0 |

### Example 3B

Results Against Pink Stain

The method used for evaluating IPBC/Ziram and IPBC/Thiram blends in plastic film was for antimicrobial activity against pink stain was according to the methodology described in ASTM E 1428-91 Standard Method for Evaluating the Performance of Antimicrobials in or on Polymeric Solids Against Staining by Streptoverticillium reticulum (A Pink Stain Organism).

Difco malt agar was prepared using manufacturers directions. Sterilized agar (30 ML) was poured into sterile plastic 100 mm Petri dishes to provide a solidified agar layer sufficient to nourish the test organism for the duration of the test.

After the agar cooled and solidified, the entire surface was uniformly streaked with inoculum of the pink stain organism Streptoverticillium reticulans ATCC 25607. Following inoculation of the agar, a sterile forceps was used to place the test specimen, previously sterilized by a brief dip in boiling water, on the agar. Each test blend was run in triplicate and the result represents the average of the three. The specimen was gently pressed on the surface of the agar to insure good contact. The Petri dishes were covered and incubated for two weeks in a 28° C. incubator.

Following incubation, samples were lifted from the surface of the agar and both sides were examined against a white background for presence of pink staining.

Degree of staining reported was determined by the area of the sample stained rather than by intensity of the color. A scale of 0 to 5 was used to indicate the amount of stain covering the specimen where 0 indicated no stain, 1 indicated a trace of stain (<10% coverage), 2 indicated a slight stain (10% to 30% coverage), 3 indicated a moderate stain (30% to 50% coverage) and 4 represented a heavy stain (>50% coverage.)

TABLE 3c

| IPBC, PPM | Ziram, PPM | Pink Stain Rating |
|---|---|---|
| 750 | 250 | 1.0 |
| 500 | 500 | 1.0 |
| 1000 | 0 | 1.3 |
| 250 | 750 | 2.0 |
| 0 | 1000 | 2.3 |

| | |
|---|---|
| Blank | 3.0 |

TABLE 3d

| IPBC, PPM | Thiram, PPM | Pink Stain Rating |
|---|---|---|
| 750 | 250 | 1.0 |
| 500 | 500 | 1.0 |
| 1000 | 0 | 1.3 |
| 250 | 750 | 2.0 |
| 0 | 1000 | 2.0 |

| | |
|---|---|
| Blank | 3.0 |

1. A biocidal composition comprising a mixture of 3-iodo-2-propynyl butyl carbamate and a dimethyldithiocarbamate.

2. The composition of claim 1 wherein the dimethyldithiocarbamate is selected from the group consisting of zinc bis(dimethyldithiocarbamate) and bis(dimethylthiocarbamoyl) disulfide.

3. A composition of claim 2 wherein the 3-iodo-2-propynyl butyl carbamate and the dimethyldithiocarbamate are present in a proportion of from about 1 part 3-iodo-2-propynyl butyl carbamate to about 25 parts dimethyldithiocarbamate to about 25 parts 3-iodo-2-propynyl butyl carbamate to about 1 part dimethyldithiocarbamate.

4. The composition of claim 3 wherein the 3-iodo-2-propynyl butyl carbamate is present in the form of an α-cyclodextrin inclusion complex.

5. The composition of claim 3 wherein the 3-iodo-2-propynyl butyl carbamate and the dimethyldithiocarbamate are present in a proportion of from about 1 part 3-iodo-2-propynyl butyl carbamate to about 9 parts dimethyldithiocarbamate to about 9 parts 3-iodo-2-propynyl butyl carbamate to about 1 part dimethyldithiocarbamate.

6. The composition of claim 5 wherein the 3-iodo-2-propynyl butyl carbamate is present in the form of an α-cyclodextrin inclusion complex.

7. A composition of claim 6 wherein the dimethyldithiocarbamate is zinc bis(dimethyldithiocarbamate).

8. The composition of claim 5 wherein the 3-iodo-2-propynyl butyl carbamate and the dimethyldithiocarbamate are present in a proportion of from about 3 parts 3-iodo-2-propynyl butyl carbamate to about 7 parts dimethyldithiocarbamate to about 7 parts 3-iodo-2-propynyl butyl carbamate to about 3 parts dimethyldithiocarbamate.

9. The composition of claim 8 wherein the 3-iodo-2-propynyl butyl carbamate is present in the form of an α-cyclodextrin inclusion complex.

10. A composition of claim 9 wherein the dimethyldithiocarbamate is zinc bis(dimethyldithiocarbamate).

11. A plastic article containing a biocidal composition comprising a mixture of an α-cyclodextrin inclusion complex of 3-iodo-2-propynyl butyl carbamate and a dimethyldithiocarbamate selected from the group consisting of zinc bis(dimethyldithiocarbamate) and bis(dimethylthiocarbamoyl)disulfide.

12. The plastic article of claim 11 wherein the 3-iodo-2-propynyl butyl carbamate and the dimethyldithiocarbamate are present in a proportion of from about 1 part 3-iodo-2-propynyl butyl carbamate to about 9 parts dimethyldithiocarbamate to about 9 parts 3-iodo-2-propynyl butyl carbamate to about 1 part dimethyldithiocarbamate.

13. The plastic article of claim 12 wherein the biocidal mixture is present from about 0.004% to about 2.0%.

14. A paint containing a biocidal composition of 3-iodo-2-propynyl butyl carbamate and a dimethyldithiocarbamate wherein the 3-iodo-2-propynyl butyl carbamate and the dimethyldithiocarbamate are present in a proportion of from about 1 part 3-iodo-2propynyl butyl carbamate to about 9 parts dimethyldithiocarbamate to about 9 parts 3-iodo-2-propynyl butyl carbamate to about 1 part dimethyldithiocarbamate.

15. The paint of claim 14 wherein the biocidal mixture is present from about 0.004% to about 2.0%.

16. The paint of claim 14 wherein the dimethyldithiocarbamate is selected from the group consisting of zinc bis(dimethyldithiocarbamate) and bis(dimethylithiocarbamoyl)disulfide.

17. The paint of claim 16 wherein the biocidal mixture is present from about 0.004% to about 2.0%.

18. A method for protecting a substrate from microbial growth which comprises treating said substrate with an effective amount of a biocidal composition comprising a mixture of 3-iodo-2-propynyl butyl carbamate and a dimethyldithiocarbamate wherein the 3-iodo-2-propynyl n-butyl carbamate and the dimethyldithiocarbamate are present in a proportion of from about 1 part 3-iodo-2-propynyl butyl carbamate to 9 parts dimethyldithiocarbamate to about 9 parts of 3 iodo-2-propynyl butyl carbamate to about 1 part zinc dimethyldithiocarbamate.
